(19) United States
(12) Patent Application Publication
(10) Pub. No.: US 2008/0160555 A1
(43) Pub. Date: Jul. 3, 2008

(54) Detecting a Microorganism Strain in a Liquid Sample

(76) Inventor: Alain Rambach, Paris (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 11/816,856

(22) PCT Filed: Feb. 21, 2006
(86) PCT No.: PCT/EP2006/060143

§ 371 Date: Aug. 22, 2007

(30) Foreign Application Priority Data
Feb. 22, 2005 (FR) 0501764

(51) Int. Cl. C12Q 1/44; C12Q 1/04

(52) U.S. Cl. 435/19; 435/34

(57) ABSTRACT

The invention concerns a medium for detecting, identifying and differentiating a microorganism strain in a liquid medium by contacting said liquid sample with a combination of chromogens substrates of enzymes expressed or not by the strain to be detected, the final coloration of the mixture being detectable in the wavelengths of the visible light.

The present invention relates to a medium for the detection, identification and differentiation of a microorganism strain in a liquid sample comprising at least two chromogenic substrates of enzymes expressed by the strain to be detected and/or another strain likely to contaminate said sample, with the final color of the sample, which is specific to the strain to be detected, being detectable at visible wavelengths when said sample is exposed to light.

The present invention also relates to a method for the detection, identification and differentiation of a microorganism strain in a liquid sample as well as a kit comprising the means necessary to implement said method.

The detection of pathogenic microorganisms and various indicators, in water in particular, has been a concern of microbiologists for many years.

Indeed, researchers have attempted to develop techniques for detecting not only indicators of fecal contamination such as E. coli, other coliforms and Enterococcus, but also pathogens such as Aeromonas.

The E. coli bacterium is a member of the coliforms. This species is highly abundant in the intestinal flora of humans and animals and is the only species known to be of strictly fecal origin. E. coli bacteria are considered to be the best indicators of fecal contamination; their presence in water indicates that the water has been contaminated by pollution of fecal origin and that other pathogenic microorganisms are likely present as well. Gastroenteritis is the most common illness associated with the ingestion of water contaminated by fecal matter. Although this illness is often benign, occasionally it can have very serious health consequences. Rarer diseases, such as hepatitis or meningitis, can also be caused by the ingestion of contaminated water.

Before the invention of chromogenic media, E. coli and other coliforms were detected by the complex study of a number of characteristics, such as lactose fermentation and acid and gas production.

Coliforms are members of the Enterobacteriaceae family (Gram⁻, non-sporulating), which comprises various genera such as Enterobacter, Klebsiella, Citrobacter and Escherichia.

It has been demonstrated that all of the microorganisms belonging to this group have β-galactosidase activity and that typical E. coli have in addition β-glucuronidase activity.

The end of the 1970's saw the gradual emergence of microorganism identification test collections using chromogenic substrates, a technique based on the fact that each microorganism strain has one or more enzyme activities (such as β-glucuronidase, β-galactosidase, α-galactosidase, β-glucosaminidase, esterases and phosphatases) likely to act on a chromogen, which then releases a chromophore giving rise to a color.

The 1990's saw the development of microorganism isolation media using precipitating chromogenic substrates.

Given that swimming-area water quality standards are given for 100 ml of water, it has become common to test a 100 ml water sample when attempting to detect the presence or absence of microorganisms or to enumerate microorganisms. However, it should be noted that current drinking water microbiological quality standards require that, among more than 60 other criteria, drinking water must not contain parasites, viruses, pathogenic bacteria or E. coli in a 100 ml sample.

Consequently, analyses must be performed throughout the water network, namely at collection points, treatment plants, reservoirs and distribution networks, in order to detect and prevent water contamination from animal or human sources.

Certain microorganism water-contamination detection methods are based on the filtration of a 100 ml sample on filter membranes that allow water to pass but that retain microorganisms. These membranes are later transferred to solid gel culture media (agar-agar or other) or to solid buffers such as paper (absorbent filter technique) or another spongy component.

In these techniques, the various strains present in the tested sample are isolated from each other, developed in the form of bacterial colonies on the surface of the aforementioned filter and then counted and identified.

These widely-used methods yield satisfactory results when implemented in combination with specific reagents. However, such methods have the disadvantages of being costly and requiring much time to implement.

Another method, which does not use gel media, is based on adding the medium directly to the liquid sample to be tested (the Colilert® test from IDEXX or the Readycult® test from Merck, for example). This method is carried out in a single container in order to obtain a qualitative result (presence/absence) or in multiple test tubes or compartments to obtain a quantitative result, as with the MPN (most probable number) method used to estimate the number of coliforms and E. coli, a method which, however, requires specific equipment as well as additional time.

Moreover, this method has the disadvantage, at least within the framework of E. coli and coliform detection, of requiring a fluorogenic substrate.

Indeed, techniques that combine a β-galactosidase substrate to detect coliforms and a glucuronidase substrate to detect E. coli generally use a chromogenic enzyme substrate to detect coliforms and a fluorogenic enzyme substrate to detect E. coli. In this case, E. coli detection requires that the sample be read under specific conditions, insofar as the technique requires that fluorescence be detected in a darkroom under UV light.

Moreover, this chromogen/fluorogen combination, by means of color and fluorescence, enables the differentiation of only two types of microorganisms defined by the respective enzymatic capacities enabling clear differentiation of one from another.

It should also be noted that techniques of the prior art using non-gel components, in which colonies are not isolated as they are with gel media, do not enable differentiation of, for example, the pathogen Aeromonas, which is, as are coliforms, a β-galactosidase-positive microorganism. Thus, it is generally proposed to add cefsulodin or another selective antimicrobial agent to the sample to be analyzed, at the risk of not being able to detect all Aeromonas and at the risk of at least partially inhibiting some E. coli.

Moreover, the chromogen/fluorogen combinations of the prior art do not enable identification of glc⁻ E. coli (atypical glucuronidase-negative E. coli) which account for approximately 5% of E. coli.

The present invention proposes to remedy the disadvantages of the prior art by the use of a combination of chromogenic enzyme substrates capable of releasing chromophores under the effect of these enzymes, said combination being selected to enable the detection, identification and differentiation of a microorganism strain in a liquid sample. Clearly, the combination of the aforementioned chromogens is to be determined as a function of the various strains of microorganisms to be detected and, more particularly, of the respective enzymatic activities of the aforementioned strains.

Indeed, the present invention relates to a medium for the detection, identification and differentiation of a microorganism strain in a liquid sample comprising:
the nutrients required for the incubation of the strain to be detected,
at least two chromogens, each being the substrate of an enzyme expressed by the strain to be detected and/or another strain likely to contaminate said sample and each releasing a chromophore under the effect of this enzyme, said chromophores contributing to the final color of the liquid mixture resulting from the addition of said medium to said liquid sample, and said color being detectable at visible wavelengths when said mixture is exposed to light.

“Strain” or “microorganism strain” means any particular microorganism species or group that is known to have common properties and that is typically identified by a common term.

Thus, within the framework of the present invention, the terms “strain” and “microorganism strain” apply in particular to E. coli strains (covering all E. coli bacteria), glc⁻ E. coli strains, typical E. coli strains (i.e., glc⁺ E. coli), coliforms other than E. coli or other than typical E. coli and bacteria of the genus Aeromonas. These terms also relate to groups of microorganism strains mentioned above such as, for example, “typical E. coli+other coliforms” or “E. coli+other coliforms.”

“Nutrients required for the incubation of the strain to be detected” means the composition of a base medium necessary for the growth of the aforementioned strain. Those persons skilled in the art know well the composition of such media and are capable of adapting them if necessary according to the specificity of certain strains. These nutrients are notably selected from the group comprising carbon, nitrogen, sulfur, phosphorus, vitamins, growth inducers, carbohydrates, salts (calcium, magnesium, manganese, sodium and potassium, for example), nutritive complexes (amino acids, blood, serum and albumin, for example) as well as peptones and animal and plant tissue extracts.

It must be stressed that the detection, identification and differentiation of a microorganism strain, within the framework of the present invention, are carried out in a non-gel mixture (comprised of the liquid sample and the inventive medium) in which microorganisms are not separated from each other, as are colonies isolated on a gel medium. Moreover, the present invention does not require the addition of fluorogenic substrates to differentiate one microorganism strain from another and the final color obtained (after an incubation period) can be seen at visible wavelengths.

In fact, after incubation, the mixture comprised of the inventive medium and the liquid sample is exposed to light, i.e., it is placed in a location where it is exposed to visible light, and the final color of this mixture is also detectable at visible wavelengths, i.e., with the naked eye. The visible spectrum is understood to extend from approximately from 400 nm to 800 nm.

Consequently, the test can be read immediately and is simplified by not requiring two successive readings. Moreover, there is no requirement for a special device such as a UV light source. Thus, the medium and the materials of the receptacle in which detection takes place, and even the contents of the sample, can either generate fluorescence or interfere with fluorescence by a quenching effect, without interfering in any way with the reading of the test.

Within the framework of the present invention, it should be noted that the chromogens used are not required for the growth of the strains to be detected. Indeed, during the incubation period, the strains develop on traditional nutrients well-known to those persons skilled in the art. Moreover, the chromogens used within the framework of the present invention may be non-precipitating, precipitating without addition or precipitating after reaction with a salt of the medium.

The inventive medium can be prepared in solid or liquid form, pre-added to the receptacle in which the test takes place or packaged in a separate container, ready to be mixed with the liquid sample to be tested.

The invention also relates to a method for the detection, identification and differentiation of a microorganism strain in a liquid sample comprising:
a) placing the liquid sample in contact with the inventive medium,
b) incubating the mixture obtained in step a) for approximately 18 to 24 hours at a temperature of approximately 34° C. to 40° C., preferably approximately 37° C.,
c) exposing the incubated mixture to light and reading the final color of said mixture at visible wavelengths, and
d) identifying the microorganism strain according to said final color.

First, within the framework of the aforementioned method, the liquid sample is placed in contact with the inventive medium either by adding the medium to the liquid sample or by adding the liquid sample to the medium already introduced into the receptacle in which the test will take place.

Next, the microorganism strain detection step is preceded by incubation of the mixture comprised of the liquid sample and the inventive medium. The incubation step can be carried out at a temperature of approximately 34° C. to 40° C., preferably 37° C., and for a period of approximately 18 to 24 hours. However, depending on the means available, those persons skilled in the art will adapt the duration of the incubation step to the temperature at which incubation is to take place.

Thus, if an incubator is not available and room temperature is below 37° C., those persons skilled in the art will extend the incubation step in order to obtain a similar result. Thus, in the absence of an incubator, the incubation step may be extended up to 48 hours or 72 hours at room temperature. In other cases, for example as a function of the richness of the medium, the incubation period could be reduced to approximately 12 to 18 hours.

Moreover, in order to increase the selectivity of the test, for the purpose of distinguishing thermotolerant coliforms (including E. coli) from other microorganism strains, incubation may be carried out for approximately 24 hours at 44-45° C., temperatures at which thermotolerant coliforms (including E. coli) are resistant.

Concerning step c) of the inventive method, there will generally be no particular step to undertake for its implementation since, for example, the test can be performed outside during daylight or inside in a room that receives direct sunlight.

It should be noted that although the inventive method can be performed completely manually, it can also be semi-automated or completely automated.

The invention also relates to a kit for implementing the inventive method comprising:
the nutrients required for the incubation of the strain to be detected,
at least two chromogens, each being the substrate of an enzyme expressed by the strain to be detected and/or another strain likely to contaminate said sample,
a receptacle to contain the liquid sample, said nutrients and said chromogens,
instructions establishing the correspondence between the final color of the mixture comprised of the liquid sample, the aforementioned nutrients and the aforementioned chromogens on one hand, and the detected strain on the other, or any other reference system enabling identification of the detected strain.

Within the framework of the present invention, the liquid or liquefied sample in which the detection, identification and differentiation of a microorganism strain takes place is preferably water, more preferentially drinking water. However, detection can also be carried out in other liquids, in particular foods such as milk, fruit juices or any other beverage.

The present invention thus makes it possible to detect and differentiate not only typical E. coli but also glucuronidase-negative (glc⁻) E. coli without having to subject all samples negative for glc to an additional indole test, which can give rise to errors for certain coliforms such as indicating that Klebsiella oxytoca is glc⁻ E. coli. The indole test is often difficult or even impossible to implement, as is the case with the Quanti-Tray® system (IDEXX) in which the sample is placed in closed, sealed compartments.

The present invention also makes it possible to distinguish E. coli from coliforms other than E. coli without confusing them with Aeromonas and without needing to add cefsulodin or another antimicrobial agent that inhibits not only Aeromonas but also partially inhibits E. coli.

Indeed, the present invention makes it possible to simultaneously detect and differentiate not only indicators of fecal contamination such as E. coli and coliforms other than E. coli but also the pathogen Aeromonas.

The detection test proposed by the present invention is essentially a qualitative test, i.e., a test that makes it possible to detect the presence or absence of a microorganism strain in a liquid sample. However, nothing prevents the inventive test from being modified into a quantitative test, for example in accordance with the MPN method.

Within the framework of the present invention, it is advisable to determine the suitable combination of chromogens for detecting the desired strain. Thus, an example of such a determination would be a chromogen that releases a chromophore that turns yellow under the effect of an enzyme expressed by coliforms other than E. coli and a chromogen that releases a chromophore that turns blue under the effect of an enzyme expressed by E. coli. Thus, if the final color of the liquid sample in which the test is performed is blue, it can be deduced that the sample is contaminated by E. coli; if the final color is yellow, it can be deduced that the sample is contaminated by coliforms other than E. coli. It should also be stressed that if the sample is contaminated by both E. coli and coliforms other than E. coli, the final color will be in the green range.

Thus, it is advisable to select chromophores as a function of the color which is sought to be observed in the case of contamination by one or the other of the microorganism strains to be detected.

The choice of chromogen combination is of primary importance but it is by no means necessary that the enzymes acting on these chromogens are specific to a microorganism strain. In certain cases, the negative characteristic for certain enzymes of the strain to be detected will be used so that the final color is representative of said strain, according to the chromophore or chromophores released.

If the liquid or liquefied sample tested contains a microorganism strain that does not have an enzyme corresponding to the substrates present in the inventive medium, and consequently no chromophore is released, the presence of said strain may, however, be detected by comparison with an uncontaminated liquid control sample. Indeed, the contaminated sample will have a milky appearance indicating microorganism growth.

Clearly, in accordance with the present invention, it is possible to envisage a large number of combinations of not only the enzymes that interact with the selected chromogens but also the chromogens themselves.

For example, among the enzymes whose activity is of use within the framework of the present invention, the following can be cited in particular: β-D-galactosaminidase, β-D-glucosaminidase, β-D-cellobiosidase, β-D-fucosidase, α-L-fucosidase, α-D-galactosidase, β-D-galactosidase, β-D-lactosidase, α-D-maltosidase, α-D-mannosidase, α-D-glucosidase, β-D-glucosidase, β-D-xylosidase, esterase, acetate esterase, butyrate esterase, carboxyl esterase, caprylate esterase, choline esterase, myo-inositol phosphatase, palmitate esterase, phosphatase, diphosphatase, aminopeptidase and sulfatase.

Concerning the chromophores which are sought to be released by the enzymatic activity of one or more microorganism strains to be detected, the following can be cited: O-nitrophenyl, P-nitrophenyl, chloro-nitrophenyl, hydroxyphenyl, nitroanilide, phenolphthalein and thymophthalein, hydroxyquinoline, cyclohexane-esculetin, dihydroxyflavone, catechol, resazurin, resofurin, VBzTM, VLM, VLPr, VQM, indoxyl, 5-bromo-4-chloro-3-indoxyl, 5-bromo-6-chloro-3-indoxyl, 6-chloro-3-indoxyl, 6-fluoro-3-indoxyl, 5-Iodo-3-indoxyl and N-methylindoxyl.

As mentioned above, the present invention makes it possible to detect, identify and differentiate the E. coli strain in a liquid sample, including when another strain is also present in said sample. However, the inventor has made the surprising observation that, even when mixed with a million times more Enterobacter coliforms, the E. coli strain could be detected after approximately 24 hours of incubation. The chromogen combination used was as follows: 5-bromo-4-chloro-3-indoxyl glucuronide, substrate for β-glucuronidase; and nitrophenyl β-galactoside, substrate for β-galactosidase. The blue-green color indicated the presence of the E. coli strain among the Enterobacter coliforms (1:1,000,000 ratio between the two strains).

The examples which follow illustrate the present invention but in no way limit its scope.

### EXAMPLES

Although the examples below represent only a few combinations of chromogens chosen as substrates for enzymes of the strains to be detected, all other combinations arising directly or indirectly from the present description also form part of the present invention.

For all of the examples which follow, the test is carried out with 100 ml of water and the step of incubating the microorganism strains to be detected was carried out with a medium comprising the following nutrients (in g/l):
peptone 5, pyruvate 1, NaCl 5, K₂HPO₄ 4, KH₂PO₄ 1, SDS 0.1, KNO₃ 0.005, tryptophan 1,
vancomycin 0.002

In the case of example 15, said medium contains neither SDS nor vancomycin.

For all of the examples which follow, incubation was carried out at 35-37° C. for approximately 24 hours.

### Example 1

The combination of chromogens is as follows:

| CHROMOGENS | ENZYME SUBSTRATES |
|---|---|
| 5-bromo-4-chloro-3-indoxyl | β-glucuronidase |
| glucuronide (XGlc) + | |
| p-nitrophenyl-α- | α-galactosidase |
| galactoside (pNP αGal) | |

ENZYMATIC ACTIVITIES: Glc/αGal

| Microorganism strain present | Chromogen combination: |
|---|---|
| in the liquid sample | XGlc + pNP αGal |
| E. coli | Green |
| E. coli + other coliforms | Green |
| Coliforms other than E. coli | Yellow |

### Example 2

The combination of chromogens is as follows:

| CHROMOGENS | ENZYME SUBSTRATES |
|---|---|
| 5-bromo-4-chloro-3-indoxyl | β-glucuronidase |
| glucuronide (XGlc) + | |
| p-nitrophenyl-α- | α-galactosidase |
| galactoside (pNP αGal) + | |
| 5-bromo-6-chloro-3-indoxyl | β-glucosidase |
| β-glucoside (Mag βGlu) | |

ENZYMATIC ACTIVITIES: Glc/αGal/βGlu

| Microorganism strain | |
|---|---|
| present in the liquid | Chromogen combination: |
| sample | XGlc + pNP αGal + Mag βGlu |
| Glc⁻ E. coli | Yellow |
| Typical E. coli | Green |
| Typical E. coli + other | Blue |
| coliforms | |
| Coliforms other than E. coli | Orange |
| Aeromonas | Mauve |

### Example 3

The same enzymes were used as in example 2 but two chromophores were reversed. The combination of chromogens is as follows:

| CHROMOGENS | ENZYME SUBSTRATES |
|---|---|
| 5-bromo-4-chloro-3-indoxyl | β-glucuronidase |
| glucuronide (XGlc) + | |
| 5-bromo-6-chloro-3-indoxyl | α-galactosidase |
| α-galactoside (Mag αGal) + | |
| p-nitrophenyl β-glucoside | β-glucosidase |
| (pNP βGlu) | |

ENZYMATIC ACTIVITIES: Glc/αGal/βGlu

| | Chromogen |
|---|---|
| | combination: |
| Microorganism strain present in | XGlc + Mag αGal + pNP |
| the liquid sample | βGlu |
| Glc⁻ E. coli | Mauve |
| Typical E. coli | Blue |
| Typical E. coli + other coliforms | Dark blue |
| Coliforms other than E. coli | Orange |
| Aeromonas | Yellow |

### Example 4

The combination of chromogens is the same as in example 2, but an inhibiter of the pathogen Aeromonas, either 0.005 g/l cefsulodin or 0.001 g/l nalidixic acid, was added to the inventive medium.

ENZYMATIC ACTIVITIES: Glc/αGal/βGlu/Aeromonas inhibitor

| | Chromogen |
|---|---|
| | combination: |
| Microorganism strain present in | XGlc + pNP αGal + Mag |
| the liquid sample | βGlu |
| glc⁻ E. coli | Yellow |
| Typical E. coli | Green |
| Typical E. coli + other coliforms | Blue |
| Coliforms other than E. coli | Orange |

### Example 5

The combination of chromogens is as follows:

| CHROMOGENS | ENZYME SUBSTRATES |
|---|---|
| 5-bromo-4-chloro-3-indoxyl | β-glucuronidase |
| glucuronide (XGlc) + | |
| p-nitrophenyl α- | α-galactosidase |
| galactoside (pNP αGal) + | |
| 5-bromo-6-chloro-3-indoxyl | β-galactosidase |
| β-galactoside (Mag βGal) | |

ENZYMATIC ACTIVITIES: Glc/αGal/βGal

| | Chromogen combination: |
|---|---|
| Microorganism strain present | XGlc + pNP αGal + Mag |
| in the liquid sample | βGal |
| E. coli | Dark blue |
| E. coli + other coliforms | Dark blue |
| Coliforms other than E. coli | Orange |
| Aeromonas | Mauve |

### Example 6

The same enzymes were used as in example 5 but two chromophores were reversed. The combination of chromogens is as follows:

| CHROMOGENS | ENZYME SUBSTRATES |
|---|---|
| 5-bromo-4-chloro-3-indoxyl | β-glucuronidase |
| glucuronide (XGlc) + | |
| 5-bromo-6-chloro-3-indoxyl | α-galactosidase |
| α-galactoside (Mag αGal) + | |
| p-nitrophenyl β- | β-galactosidase |
| galactoside (pNP βGal) | |

ENZYMATIC ACTIVITIES: Glc/αGal/βGal

| Microorganism strain | |
|---|---|
| present in the liquid | Chromogen combination: |
| sample | XGlc + Mag αGal + pNP βGal |
| E. coli | Dark blue |
| E. coli + other coliforms | Dark blue |
| Coliforms other than E. coli | Orange |
| Aeromonas | Yellow |

### Example 7

The combination of chromogens is the same as in example 5, but an inhibiter of the pathogen Aeromonas, either 0.005 g/l cefsulodin or 0.001 g/l nalidixic acid, was added to the inventive medium.

ENZYMATIC ACTIVITIES: Glc/αGal/βGal/Aeromonas inhibitor

| Microorganism strain | |
|---|---|
| present in the liquid | Chromogen combination: |
| sample | XGlc + pNP αGal + Mag βGal |
| E. coli | Dark blue |
| E. coli + other coliforms | Dark blue |
| Coliforms other than E. coli | Orange |

### Example 8

The combination of chromogens is as follows:

| CHROMOGENS | ENZYME SUBSTRATES |
|---|---|
| 5-bromo-4-chloro-3-indoxyl | β-glucuronidase |
| glucuronide (XGlc) + | |
| p-nitrophenyl β- | β-galactosidase |
| galactoside (pNP βGal) | |

ENZYMATIC ACTIVITIES: Glc/βGal

| Microorganism strain present | Chromogen combination: |
|---|---|
| in the liquid sample | XGlc + pNP βGal |
| E. coli | Blue-green |
| E. coli + other coliforms | Blue-green |
| Coliforms other than E. coli | Yellow |

### Example 9

The enzymes are the same as in example 8 but one chromophore is different. The combination of chromogens is as follows:

| CHROMOGENS | ENZYME SUBSTRATES |
|---|---|
| 5-bromo-4-chloro-3-indoxyl | β-glucuronidase |
| glucuronide (XGlc) + | |
| 5-bromo-6-chloro-3-indoxyl β- | β-galactosidase |
| galactoside (Mag βGal) | |

ENZYMATIC ACTIVITIES: Glc/βGal

| Microorganism strain present | Chromogen combination: |
|---|---|
| in the liquid sample | XGlc + Mag βGal |
| E. coli | Dark blue |
| E. coli + other coliforms | Dark blue |
| Coliforms other than E. coli | Mauve |

### Example 10

The combination of chromogens is as follows:

| CHROMOGENS | ENZYME SUBSTRATES |
|---|---|
| 5-bromo-4-chloro-3-indoxyl | β-glucuronidase |
| glucuronide (XGlc) + | |
| p-nitrophenyl β- | β-galactosidase |
| galactoside (pNP βGal) + | |
| 5-bromo-6-chloro-3-indoxyl | β-glucosidase |
| β-glucoside (Mag βGlu) | |

ENZYMATIC ACTIVITIES: Glc/βGal/βGlu

| | Chromogen |
|---|---|
| | combination: |
| Microorganism strain present in | XGlc + pNP βGal + |
| the liquid sample | Mag βGlu |
| Glc⁻ E. coli | Yellow |
| Typical E. coli | Green |
| Typical E. coli + other coliforms | Blue |
| Coliforms other than E. coli | Orange |
| Aeromonas | Mauve |

### Example 11

The enzymes are the same as in example 10 but two chromophores were reversed. The combination of chromogens is as follows:

| CHROMOGENS | ENZYME SUBSTRATES |
|---|---|
| 5-bromo-4-chloro-3-indoxyl | β-glucuronidase |
| glucuronide (XGlc) + | |
| 5-bromo-6-chloro-3- | β-galactosidase |
| indoxyl β-galactoside (Mag | |
| βGal) + | |
| p-nitrophenyl β-glucoside | β-glucosidase |
| (pNP βGlu) | |

ENZYMATIC ACTIVITIES: Glc/βGal/βGlu

| | Chromogen combination: |
|---|---|
| Microorganism strain present | XGlc + Mag βGal + pNP |
| in the liquid sample | βGlu |
| Glc⁻ E. coli | Mauve |
| Typical E. coli | Blue |
| Typical E. coli + other | Dark blue |
| coliforms | |
| Coliforms other than E. coli | Orange |
| Aeromonas | Yellow |

### Example 12

The combination of chromogens is the same as in example 10, but an inhibiter of the pathogen Aeromonas, either 0.005 g/l cefsulodin or 0.001 g/l nalidixic acid, was added to the inventive medium.

ENZYMATIC ACTIVITIES: Glc/βGal/βGlu/Aeromonas inhibitor

| Microorganism strain | Chromogen combination: |
|---|---|
| present in the liquid | XGlc + pNP βGal + |
| sample | Mag βGlu |
| Glu⁻ E. coli | Yellow |
| Typical E. coli | Green |
| Typical E. coli + other | Blue |
| coliforms | |
| Coliforms other than E. coli | Orange |

### Example 13

The combination of chromogens is as follows:

| CHROMOGENS | ENZYME SUBSTRATES |
|---|---|
| 5-bromo-4-chloro-3-indoxyl | β-glucuronidase |
| glucuronide (XGLc) + | |
| p-nitrophenyl β-glucoside | β-glucosidase |
| (pNP βGlu) | |

ENZYMATIC ACTIVITIES: Glc/βGlu

| Microorganism strain | |
|---|---|
| present in the liquid | Chromogen combination: |
| sample | XGlc + pNP βGlu |
| E. coli | Blue |
| E. coli + other coliforms | Blue-green |
| Coliforms other than E. coli | Yellow |

### Example 14

The enzymes are the same as in example 13 but one chromophore is different. The combination of chromogens is as follows:

| CHROMOGENS | ENZYME SUBSTRATES |
|---|---|
| 5-bromo-4-chloro-3-indoxyl | β-glucuronidase |
| glucuronide (XGlc) + | |
| 5-bromo-6-chloro-3-indoxyl | β-glucosidase |
| β-glucoside (Mag βGlu) | |

ENZYMATIC ACTIVITIES: Glc/βGlu

| Microorganism strain | |
|---|---|
| present in the liquid | Chromogen combination: |
| sample | XGlc + Mag βGlu |
| E. coli | Blue |
| E. coli + other coliforms | Dark blue |
| Coliforms other than E. coli | Mauve |

### Example 15

The combination of chromogens is as follows:

| CHROMOGENS | ENZYME SUBSTRATES |
|---|---|
| 5-bromo-4-chloro-3-indoxyl | β-glucuronidase |
| glucuronide (XGlc) + | |
| 5-bromo-6-chloro-3- | β-galactosidase |
| indoxyl β-galactoside (Mag | |
| βGal) + | |
| p-nitrophenyl β-glucoside | β-glucosidase |
| (pNP βGlu) | |

ENZYMATIC ACTIVITIES: Glc/βGal/βGlu

| Microorganism strain | Chromogen combination: |
|---|---|
| present in the liquid | XGlc + Mag βGal + |
| sample | pNP βGlu |
| E. coli | Blue |
| Coliforms other than E. coli | Orange |
| Enterococcus | Yellow |

1. A medium for the detection, identification and differentiation of a microorganism strain chosen among the group comprising E. coli and/or coliforms other than E. coli, typical E. coli and/or coliforms other than typical E. coli, glc⁻ E. coli and Aeromonas, in a liquid sample likely to contain at least one of the aforementioned strains, said medium comprising:
the nutrients required for the incubation of the strain to be detected,
at least two chromogens, each being the substrate of an enzyme expressed by the strain to be detected and/or another strain likely to contaminate said sample and each releasing a chromophore under the effect of this enzyme,
said chromophores contributing to the final color of the liquid mixture resulting from the addition of said medium to said liquid sample,
and said color being detectable at visible wavelengths when said mixture is exposed to light.

2. A medium according to claim 1, wherein the liquid sample is water, preferably drinking water.

3. A medium according to claim 1 or claim 2, wherein said enzyme is chosen among the group comprising β-D-galactosaminidase, β-D-glucosaminidase, β-D-cellobiosidase, β-D-fucosidase, α-L-fucosidase, α-D-galactosidase, β-D-galactosidase, β-D-lactosidase, α-D-maltosidase, α-D-mannosidase, α-D-glucosidase, β-D-glucosidase, β-D-xylosidase, esterase, acetate esterase, butyrate esterase, carboxyl esterase, caprylate esterase, choline esterase, myo-inositol phosphatase, palmitate esterase, phosphatase, diphosphatase, aminopeptidase and sulfatase.

4. A medium according to one of the claims 1 to 3, wherein said chromophore is chosen among the group comprising O-nitrophenyl, P-nitrophenyl, chloro-nitrophenyl, hydroxyphenyl, nitroanilide, phenolphthalein and thymophthalein, hydroxyquinoline, cyclohexane-esculetin, dihydroxyflavone, catechol, resazurin, resofurin, VBzTM, VLM, VLPr, VQM, indoxyl, 5-bromo-4-chloro-3-indoxyl, 5-bromo-6-chloro-3-indoxyl, 6-chloro-3-indoxyl, 6-fluoro-3-indoxyl, 5-Iodo-3-indoxyl and N-methylindoxyl.

5. A method for the detection, identification and differentiation of a microorganism strain chosen among the group comprising E. coli and/or coliforms other than E. coli, typical E. coli and/or coliforms other than typical E. coli, glc⁻ E. coli and Aeromonas, in a liquid sample likely to contain at least one of the aforementioned strains, said method comprising:
a) placing the liquid sample in contact with a medium according to one of the claims 1 to 4,
b) incubating the mixture obtained in step a) for approximately 18 to 24 hours at a temperature of approximately 34° C. to 40° C., preferably approximately 37° C.,
c) exposing the incubated mixture to light and reading the final color of said mixture at visible wavelengths, and
d) identifying the microorganism strain according to said final color.

6. A method according to claim 5, wherein the liquid sample is water, preferably drinking water.

7. A kit for the implementation of the method according to claim 5 or claim 6, comprising:
the nutrients required for the incubation of the strain to be detected,
at least two chromogens, each being the substrate of an enzyme expressed by the strain to be detected and/or another strain likely to contaminate said sample,
a receptacle to contain the liquid sample, said nutrients and said chromogens,
instructions establishing the correspondence between the final color of the mixture comprised of the liquid sample, the aforementioned nutrients and the aforementioned chromogens on one hand, and the detected strain on the other, or any other reference system enabling identification of the detected strain.

8. A kit according to claim 7, wherein the liquid sample is water, preferably drinking water.
